(19) United States
(12) Patent Application Publication
(10) Pub. No.: US 2021/0106745 A1
(43) Pub. Date: Apr. 15, 2021

(54) BLOOD-DEGASSING APPARATUS AND BLOOD-TREATMENT SYSTEM

(71) Applicant: RHEINISCH-WESTFÄLISCHE TECHNISCHE HOCHSCHULE (RWTH) AACHEN, Aachen (DE)

(72) Inventor: Ahmed Ali KASHEFI-KHORASANI, Aachen (DE)

(21) Appl. No.: 16/970,547

(22) PCT Filed: Mar. 20, 2019
(86) PCT No.: PCT/EP2019/056974

§ 371 Date: Aug. 17, 2020

(30) Foreign Application Priority Data
Mar. 22, 2018 (DE) 10 2018 002 385.1

(51) Int. Cl. A61M 1/36; A61M 1/32

(52) U.S. Cl. CPC A61M 1/3627; A61M 1/32

(57) ABSTRACT

The invention relates to a degassing devive (25) for degassing blood, comprising a blood chamber (1, 1a, 1b) with a blood inlet (2) and a blood outlet (3), via which blood can be guided/is guided through the blood chamber (1, 1a, 1b), at least one underpressure chamber (11) with an underpressure attachment (8) via which the at least one underpressure chamber (11) is provided with an underpressure, at least one semipermeable membrane (4, 4a), which is arranged between the at least one underpressure chamber (11) and the blood chamber (1), wherein the blood chamber (1) comprises a first and a second sub-chamber (1a, 1b) which are arranged next to each other in a direction perpendicular to the direction of gravity, and a bridging region (9) which connects the two subchambers (1a, 1b) at their upper ends, wherein the blood inlet (2) is arranged in the lower region of the first subchamber (1a), and the blood outlet (3) is arranged in the lower region of the second subchamber (1b). The invention also relates to a system for extracorporeal treatment of blood.

The invention relates to a blood-degassing apparatus, in particular for removing dissolved gas or gas bubbles from blood. The invention also relates to a system for the extracorporeal treatment of blood, comprising a blood-drawing implement, in particular a blood-drawing cannula, and a blood-return implement, in particular a blood return cannula, at least one blood-conducting hose, at least one blood pump and an oxygenator being between the implements, in particular so that blood can be pumped through the oxygenator using the at least one blood pump. The invention also relates to a system and to a method for the extracorporeal purification of blood withdrawn from a human body or an animal body, in which gas, and preferably gas bubbles, present in the blood is or are eliminated.

Degassing of blood shall be understood to mean for example that gas dissolved in the blood is removed from the blood. Dissolved gas is for example gas that is bound to the hemoglobin, such as carbon dioxide or also carbon monoxide. Such dissolved gases are thus not present in bubble form in the blood. These two types of gases are undesirable in the blood for example since they limit supply of oxygen to the patient.

It is known in the related art, for example, to remove dissolved carbon dioxide, or also carbon monoxide, from blood by having an increased partial pressure of oxygen act on the blood, so that the carbon dioxide or carbon monoxide is exchanged with oxygen by diffusion. So-called oxygenators are typically used for eliminating the carbon dioxide.

These are essentially apparatuses comprising a chamber holding a bundle of semipermeable fiber tubes through which oxygen flows for oxygenation, while the blood flows around the fiber tubes. As a result of the high partial pressure of oxygen, and thus low partial pressure of carbon dioxide, the carbon dioxide is displaced through the semipermeable fiber tubes from the blood present around the fiber tubes by the oxygen, thereby creating a gas exchange.

Carbon monoxide is eliminated from blood for example in that a patient in question undergoes what is known as hyperbaric oxygen therapy in a hyperbaric chamber, or else that at least blood withdrawn from the patient is treated with oxygen overpressure and subsequently returned to the patient.

One disadvantage of such methods is that the gas exchange, and thus the degassing of the undesirable gas from the blood, only takes place by the action of the increased partial pressure of oxygen, as a result of which an excess supply of oxygen has to be provided to eliminate a certain amount of dissolved gas from the blood. Moreover, it is disadvantageous that the oxygen fractions not participating in the gas exchange are usually lost to the surrounding area. In addition, the diffusion processes are slow. At the same time, eliminating carbon monoxide for example from blood is a time-critical process and has to be carried out as quickly as possible.

Degassing within the meaning of the invention shall also be understood to mean that gases not dissolved in the blood, that is for example gases present in the blood in the form of bubbles, are removed from the blood. These gases, such as ambient air, can be present, for example, in blood that is drawn off during surgery from the surgical site by a pump. This can take place for example in addition to the use of the system mentioned at the outset that is used to oxygenate the blood of a patient in a circuit during surgery. In addition, treatment methods can provide that oxygen is conducted in the form of bubbles through the blood so as to achieve a more effective exchange between oxygen and a gas dissolved in the blood, such as carbon dioxide, or particularly carbon monoxide.

To the extent that such blood containing bubbles is to be returned to the patient's body, there is a need to first remove the gas bubbles from the blood. According to the existing related art, an elimination of bubble-shaped gases from blood is carried out for example by defoamers that essentially comprise a filter/sponge through which the blood flows, thereby retaining the gas bubbles. Such defoamers have the disadvantage that shearing forces act on the blood as the blood flows through a filter/sponge, which can result in thrombogenesis. In addition, some gas bubbles can also find their way through the defoamers.

It is thus the object of the invention to provide a blood-degassing apparatus, a system of the above-described type, and a method for degassing blood, and for each higher effectiveness is achieved and in particular faster treatment option and/or gentler and/or safer treatment option is achieved. Particularly preferably, a degassing apparatus of the invention must have an oxygen-conserving design, in particular if it is used for oxygenating blood at the same time.

This object is achieved by a degassing apparatus comprising a blood chamber having a blood inlet and a blood outlet, between which blood can be/is conducted through the blood chamber and having at least one subatmospheric-pressure chamber including a subatmospheric-pressure connection through which the at least one subatmospheric-pressure chamber is evacuated, and having at least one semipermeable membrane between the at least one subatmospheric-pressure chamber and the blood chamber. Accordingly, the volumes of the blood chamber and of the subatmospheric-pressure chamber are separated from one another by at least one semipermeable membrane. The object is also achieved by a method of the above-described type in which the dissolved gas is eliminated or gas bubbles are eliminated by conducting the blood along one side of a semipermeable membrane, and a subatmospheric pressure is applied to the other side of the semipermeable membrane.

A subatmospheric pressure within the meaning of the invention shall be understood to mean such a pressure that is at least lower than the atmospheric ambient pressure in the surrounding area in which the degassing apparatus or the system is located or the method is carried out. The subatmospheric pressure in the subatmospheric-pressure chamber is preferably lower than the atmospheric ambient pressure by at least 100 mbar, more preferably by at least 200 mbar, still more preferably by at least 300 mbar, still more preferably by at least 400 mbar, still more preferably by at least 600 mbar, and still more preferably by at least 800 mbar.

To the extent that gas bubbles, for example from the ambient air, or also from pure or medical oxygen, are to be eliminated from the blood, it is obvious that any subatmospheric pressure that is lower than the ambient pressure causes a reduction in, and preferably a complete elimination of, the gas bubbles, since ambient pressure is always present in the gas bubbles.

If the apparatus or the method is used to eliminate gases dissolved in the blood, the subatmospheric pressure in the subatmospheric-pressure chamber is preferably selected such that the partial pressure of the gas to be eliminated from the blood in the subatmospheric-pressure chamber is lower than the partial pressure of this gas in the blood. This is also achieved, in particular, by the described subatmospheric pressure ranges.

Both in the case of gas bubbles in the blood and in the case of gases dissolved in the blood, the pressure differences for the purpose of pressure equalization cause the gas from the blood to cross the semipermeable membrane and enter the subatmospheric-pressure chamber where it is drawn off, for example using a vacuum pump.

This method is considerably faster compared to the related prior art, in particular since this degassing according to the invention does not result in an exchange of the gas to be removed with another gas, such as oxygen, even though such an exchange is also possible with the invention.

In one refinement, for example, the invention can provide that an oxygen atmosphere is present in the subatmospheric-pressure chamber, for example in that oxygen, and in particular pure or medical oxygen, is introduced into the subatmospheric-pressure chamber by an oxygen supply, and this oxygen is drawn off by the subatmospheric pressure source from the subatmospheric-pressure chamber. In this way, a subatmospheric pressure is ensured, however with an overwhelming presence of oxygen in the subatmospheric-pressure chamber that can pass through the at least one semipermeable membrane into the blood.

According to the invention, there is the option to set, in the subatmospheric-pressure chamber, such a partial pressure of the gas that is not desired in the blood which causes this undesirable gas to leave the blood, that is, is lower than the partial pressure of this gas in the blood, an oxygen is available as an exchange partner so as to simultaneously effectuate oxygenation of the blood.

Such a procedure is particularly fast in terms of eliminating the undesirable, for example dissolved gas or also gas bubbles, from blood. In addition, this procedure is gentler for the blood since, in contrast to the related art, the blood does not have to cross a filter/sponge, but the gas passes through the at least one membrane here. As a result, no shear forces act on the blood.

The invention can provide that the blood is subjected to further oxygenation before the blood thus treated according to the invention is returned, for example using a conventional oxygenator. For this purpose, such an oxygenator can be connected in the flow of a degassing apparatus according to the invention, and in particular be downstream of the degassing apparatus.

The described subatmospheric pressure is provided by a subatmospheric pressure source that is connected to the subatmospheric-pressure connection of the subatmospheric-pressure chamber. For example, such a subatmospheric pressure source can be formed by a vacuum pump, such as a diaphragm pump or also a rotary vane pump. The term “vacuum pump” here does not imply the capability to create an ideal vacuum, but only to be able to generate a subatmospheric pressure below that of the surrounding area.

To the extent that the apparatus comprises multiple subatmospheric-pressure chambers, the invention may provide that each subatmospheric-pressure chamber of the apparatus is connected to a respective subatmospheric pressure source, for example a vacuum pump, or also that multiple subatmospheric-pressure chamber of all, or all subatmospheric-pressure chambers, are connected to a common subatmospheric pressure source.

A semipermeable membrane is preferably understood to mean a membrane that is permeable to gas, such as carbon dioxide and/or carbon monoxide and/or oxygen, and is impermeable to blood or the constituents thereof (such as the blood plasma). Such a semipermeable membrane may for example be a silicone membrane or for example be made of the polypropylene or, generally speaking, of a hydrophobic polymer material. A membrane can have pores for example through which a convective gas transport can take place. Such a semipermeable membrane preferably has a pore size of less than 200 nanometers. A semipermeable membrane may also be designed without pores, and the gas transport then takes place by diffusion through the membrane.

The preferred refinements furthermore described below can be used both during a degassing of gas dissolved in blood and of gas bubbles in blood, the latter application being furthermore preferred.

One refinement can provide for example that the blood chamber comprises an upstream and a downstream subchamber that are next to one another in a direction perpendicular to the direction of gravity, and a bridging subchamber that connects the two subchambers at the upper ends thereof, and the blood inlet is in the lower region of the upstream subchamber, and the blood outlet is in the lower region of the downstream subchamber.

Such an embodiment essentially results in a U-shaped flow of the blood flowing through the blood chamber, namely upwardly, counter to the direction of gravity in the upstream subchamber then in particular at least substantially in the horizontal direction in the bridging subchamber between the subchambers, and then downward in the direction of gravity in the downstream subchamber whence the blood exits.

Since the blood chamber according to the invention the at least one semipermeable membrane delimiting at least one subatmospheric-pressure chamber degasses the blood on the way thereof through the blood chamber, and in particular gas bubbles are eliminated.

In the event that bubbles are still present in the blood in the flow direction downstream of the at least one membrane, the flow in the direction of gravity in the downstream subchamber causes the remaining gas bubbles, by virtue of the buoyancy thereof, to float upward counter to the flow and to not leave the apparatus and be eliminated through the action of further subatmospheric pressure via the membrane.

According to the invention, it is not absolutely necessary for the blood to flow exactly in or counter to the direction of gravity. What is essential is that the apparatus is configured so that the blood in the upstream subchamber flows at least on average parallel counter to the direction of gravity and in the downstream subchamber flows at least on average parallel in the direction of gravity.

A preferred refinement can provide that at least one subregion of the upper wall region of the bridging subchamber is formed by a semipermeable membrane, and in particular a semipermeable flat membrane whose one side faces the volume of the blood chamber and whose opposite side faces the volume of the subatmospheric-pressure chamber, and in particular one of several subatmospheric-pressure chambers. This semipermeable membrane may be the only semipermeable membrane of the degassing apparatus, or one of several. For example, further semipermeable membranes can be in at least one or both subchambers.

The use of at least one semipermeable membrane, and in particular a flat membrane, in the bridging subchamber has the advantage that gas bubbles by virtue of their buoyancy collect at this membrane and reside there or by virtue of the buoyancy thereof flow back there and are thus effectively removed at this site since the subatmospheric pressure is acting on the other face of the membrane. The membrane can preferably form the highest point in the bridging subchamber in the degassing apparatus, or at least include the highest point, at which the blood flows past on the way thereof through the blood chamber.

In this preferably U-shaped design of the blood chamber, but also in all other possible configurations of the degassing apparatus, a further preferred design can be that an oxygen-bubble-generating gassing device, in particular a frit filter exposed to oxygen, is in the region of the blood inlet of the blood chamber, in particular inside the blood chamber, and in particular of the blood-inlet-side upstream subchamber.

By supplying oxygen in the form of bubbles, effective oxygenation of the blood can thus be created, and the oxygen bubbles are thereafter eliminated from the blood at the at least one semipermeable membrane as a result of the subatmospheric pressure in the subatmospheric-pressure chamber, but preferably no degassing of the oxygen dissolved in the blood takes place.

It is preferably provided for this purpose to set the pressure in the subatmospheric-pressure chamber lower than the ambient pressure, however in such a way that the partial pressure of oxygen in the subatmospheric-pressure chamber is greater than the partial pressure of the oxygen dissolved in the blood. This can in turn be achieved for example by generating the subatmospheric pressure in an oxygen atmosphere inside the subatmospheric-pressure chamber that in particular is achieved in the manner as described above.

In all possible embodiments of the blood chamber, and particularly preferably in the described U-shaped design, according to the invention a bundle of semipermeable fiber tubes is contained in the blood chamber, and in particular the blood-outlet-side downstream subchamber or in both subchambers of the described embodiment, the outer surfaces of the fiber tubes being contacted by blood and their interiors being connected to or forming a subatmospheric-pressure chamber, and in particular one of several subatmospheric-pressure chambers. Each semipermeable fiber tube forms a semipermeable membrane, in particular formed into a tube. In this embodiment, the number of the semipermeable membranes in the blood chamber, or in one or both subchambers, is identical to the number of the fiber tubes in this chamber.

As an alternative, the invention can provide in all possible embodiments of the blood chamber, and particularly preferably in the described U-shaped design, that a bundle of semipermeable fiber tubes is in at least one subatmospheric-pressure chamber, their outer surfaces being acted upon by subatmospheric pressure, and their interiors being filled by blood, and the tube interiors form at least a portion of the blood chamber, and in particular a portion of at least one subchamber, or form same as a whole.

The degassing apparatus as a whole or one subchamber or both subchambers of the above-described embodiment can thus comprise for example a commercially available oxygenator or also a dialyzer, and in particular be formed of it, in these described two alternative embodiments. The two described embodiments then essentially differ in that the blood either flows around the outsides of the fiber tubes, and the subatmospheric pressure is present inside the fiber tubes, or that the blood flows inside the fiber tubes and the subatmospheric pressure is present around the outsides of the fiber tubes.

As was already mentioned at the outset, the invention can provide that an oxygen supply opens into the at least one subatmospheric-pressure chamber that in particular is configured to supply the subatmospheric-pressure chamber continuously with oxygen, while maintaining a subatmospheric pressure.

The system according to the invention for the extracorporeal treatment of blood comprises a blood-drawing implement, in particular a blood-drawing cannula, and a blood-return implement, in particular a blood return cannula, and at least one blood-conducting hose, at least one blood pump and an oxygenator are between the implements, in particular so that blood can be pumped through the oxygenator using the at least one blood pump. Such a system is known for supplying the blood of a patient with oxygen during surgery, for which purpose it is conducted through an oxygenator, and in particular one of the type described at the outset.

According to the invention, it is now furthermore provided that this system also comprises a degassing apparatus according to one of the above-described embodiments, having a blood outlet that opens into the at least one blood-conducting hose between the implements.

In this way, it is possible, by the degassing apparatus, to degas blood in the system, for example of blood that is pumped in the circuit of the system, or also of blood that was aspirated from a surrounding area of the surgical site by a pump, for example a peristaltic pump.

For example, the blood conducted in the circuit can be degassed using the degassing apparatus in the sense that carbon dioxide and/or carbon monoxide dissolved therein is degassed. For this purpose, the degassing apparatus can for example be in the circuit upstream of the oxygenator.

As an alternative, or also in addition, to the preceding embodiment, the blood drawn off from a surrounding area of a surgical site can be freed of gas bubbles using the degassing apparatus. The blood thus freed of bubbles can then be supplied to the blood-conducting tube of the system that conducts the blood of the patient in the circuit through the oxygenator. The blood freed of bubbles is thus returned to the patient.

The embodiment of the system is particularly preferred when this system is configured to conduct a subflow of the overall blood flowing in the circuit between the implements (blood-drawing implement and blood-return implement) through the degassing apparatus. This has the advantage that the degasser is continuously integrated into the blood circuit of the system, in particular thus not only at the times when degassing of bubbles from blood is needed. In this way, blood is prevented from stagnating in the degasser.

A refinement of the system can provide that an inflow line is included through blood loaded with gas bubbles can be aspirated from a surrounding area of the surgical site, in particular using a blood pump in the inflow line, and this inflow line opens into the blood inlet of the degassing apparatus. One embodiment can then provide for example that the blood outlet of the degassing apparatus opens somewhere into the tube of the system in which the blood is pumped in the circuit, that is for example also upstream of the oxygenator.

The invention, in contrast, particularly preferably provides that the blood outlet of the degassing apparatus opens into the blood inlet of the oxygenator. In this way, the degassed blood is enriched with oxygen in the oxygenator prior to being returned to the patient.

According to a particularly preferred embodiment of the invention, the system is configured to control the generation of subatmospheric pressure in the at least one subatmospheric-pressure chamber of the degassing apparatus as a function of the operation of the blood pump of the surgical site blood-drawing implement, thus preferably of the blood pump in the inflow line, in particular in such a way that a subatmospheric pressure is automatically generated in the at least one subatmospheric-pressure chamber as soon as, or before, the blood pump of the surgical site aspirating implement starts to operate, more preferably wherein an ambient pressure is present in the at least one subatmospheric-pressure chamber as soon as, or after, this blood pump has stopped operating.

In this way, subatmospheric pressure is automatically present in the degassing apparatus whenever blood is drawn off from the surgical site, but not when no aspirating is taking place.

In all the described embodiments, a method for the extracorporeal purification of blood that was withdrawn from a human body or an animal body is thus characterized in that gas, preferably gas bubbles, present in the blood is or are eliminated by conducting the blood along one side of a semipermeable membrane, and a subatmospheric pressure, and preferably an oxygen atmosphere that is under subatmospheric pressure, is applied to the other side of the semipermeable membrane.

Preferred embodiments of the invention are described below with reference to the figures in which:

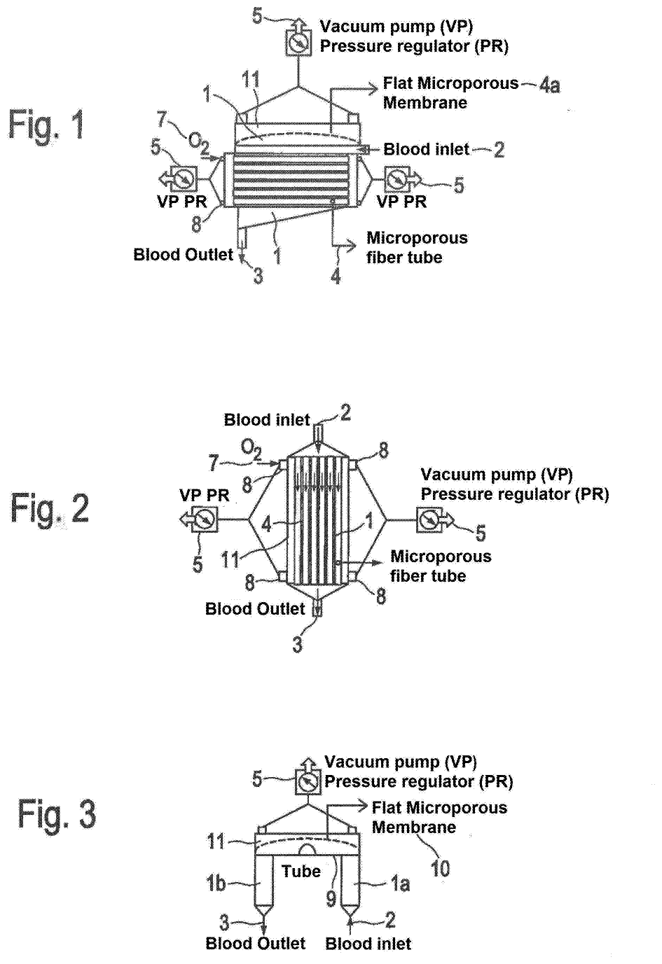

FIG. 1 shows a first possible embodiment of a degassing apparatus according to the invention, having a blood chamber 1 through which blood can be conducted from a blood inlet 2 to a blood outlet 3.

A plurality of semipermeable fiber tubes 4 are in the blood chamber 1, the outsides of the fiber tubes being in contact with the blood in the blood chamber 1, and the tube interiors being placed under subatmospheric pressure by at least one vacuum pump 5. For this purpose, all open ends of the fiber tubes can collectively open into a chamber that is at subatmospheric pressure that here, in particular, is placed under subatmospheric pressure via at least one subatmospheric-pressure connection 8, two subatmospheric-pressure connections being shown here, per vacuum pump 5. The sum of all inner volumes of the fiber tubes 4 and of the described chamber into which these open forms the subatmospheric-pressure chamber within the meaning of the invention. Each fiber tube 4 forms a semipermeable membrane.

In addition, here a semipermeable, and preferably microporous, flat membrane 4a is in the blood inlet region of the blood chamber 1 and separates the blood chamber 1 from a further subatmospheric-pressure chamber 11 evacuated by a further vacuum pump 5. The flat membrane 4a can have a longitudinal extension that corresponds to the lengths of the fiber tubes 4.

It is furthermore shown here that oxygen is introduced into the subatmospheric-pressure chamber via an oxygen supply 7 so that the subatmospheric atmosphere predominantly contains oxygen, having the advantages described above.

The apparatus shown in FIG. 1 can be formed by an oxygenator or a dialyzer, to which a vacuum pump 5 is connected, for example at the gas outlet or outlets 8. This gas outlet 8 thus forms the subatmospheric-pressure connection 8.

FIG. 2 shows a preferred embodiment in which the blood supply 2 and the connections of the vacuum pump 5 and the oxygen supply 7 are reversed. Accordingly, compared to FIG. 1, the blood moves through inside the fiber tubes 4 here, and the subatmospheric pressure is present on the outside of the fiber tubes 4. Otherwise, the function is identical to FIG. 1. In this respect, the blood chamber 1 here is formed by the sum of the inner volumes of the fiber tubes 4, and the subatmospheric-pressure chamber 11 is formed by surrounding volume that in particular is delimited by the outer housing of the apparatus.

FIG. 3 shows an embodiment in which the blood chamber 1 of the degassing apparatus is divided into two subchambers 1a and 1b offset transversely to the direction of gravity, that is horizontally, next to one another, and the blood flow in each of the subchambers takes place on average parallel to the direction of gravity, namely upwardly from the blood inlet opposite the direction of gravity in the subchamber 1a, and in the direction of gravity to the blood outlet 3 in the subchamber 1b.

The upper ends of the subchambers 1a and 1b are connected by a substantially horizontal bridging subchamber 9 in which the blood is transferred basically horizontally from the upstream subchamber 1a to the downstream subchamber 1b.

This bridging subchamber 9 is divided by a semipermeable membrane 10, for example a flat membrane, into a lower region that is part of the blood chamber, that is through which blood flows, and an upper region that forms the subatmospheric-pressure chamber 11 to which the vacuum pump 5 is connected.

As a result of the subatmospheric pressure, the membrane 10 is upwardly curved and, in the upper region of the curvature, forms the highest point of blood flow in the blood chamber. Existing gas bubbles will thus collect here, due to buoyancy, and are eliminated. Gas bubbles not eliminated continue to be prevented from flowing out of the blood chamber in that the gas bubbles, by virtue of the buoyancy thereof, are prevented from flowing downward in the downstream subchamber 1b, and thus remain in the blood chamber until these are completely eliminated.

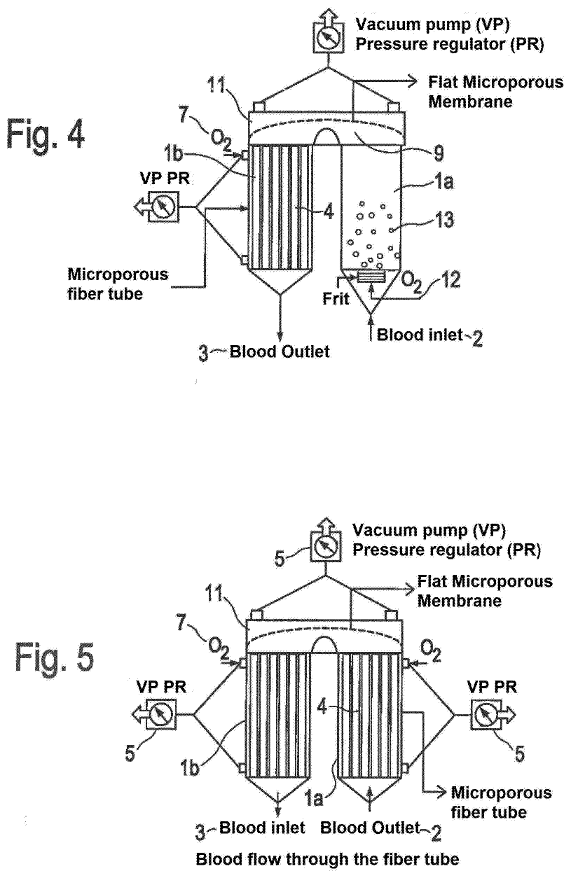

FIG. 4 shows a refinement of the embodiment of FIG. 3. In this embodiment, semipermeable fiber tubes 4 are at least in the downstream subchamber 1b as already described with regard to FIG. 1. Subatmospheric pressure is applied to them internally, and blood flows outside around them.

Accordingly, this degassing apparatus comprises a blood chamber including the two subchambers 1a and 1b and the bridging subchamber 9, as well as two subatmospheric-pressure chambers, namely one subatmospheric-pressure chamber 11 above the membrane 10 in the bridging subchamber 9, and one that is formed of the sum of the inner volumes of the fiber tubes 4, and where applicable also in a chamber region into which the open ends of the fiber tubes 4 lead.

Both subatmospheric-pressure chambers can be evacuated by separate vacuum pumps or another subatmospheric pressure source.

It may also be in the embodiments of FIGS. 3 and 4 that oxygen is supplied to the subatmospheric-pressure chamber(s) so as to form therein an atmosphere that predominantly, or exclusively, contains oxygen.

FIG. 4 furthermore shows that there is the option of integrating an oxygen supply 12 into the region of the blood inlet 2 so that oxygen bubbles 13 for oxygenation of the blood can be formed. For example, a frit filter passing incident oxygen flow may be provided here. Remaining oxygen bubbles are then removed again in the upper transition region.

FIG. 5 shows an embodiment in which semipermeable fiber tubes 4 are provided both in the upstream subchamber 1a and in the downstream subchamber 1b. Otherwise, the embodiment is identical to that of FIG. 4, however without oxygen supply into the blood chamber that, however, could also be provided.

This embodiment thus results in a total of 3 subatmospheric-pressure chambers that can be evacuated by three vacuum pumps 5.

Instead of the blood flowing around the fiber tubes 4, and the subatmospheric pressure prevailing in the fiber tubes 4, it is also possible in the embodiments of FIGS. 4 and 5 for the blood to flow in the fiber tubes 4, and the subatmospheric pressure to be present around the outside of the fiber tubes. The latter is the preferred embodiment of the designs of FIGS. 4 and 5. This does not result in any visual difference in FIGS. 4 and 5.

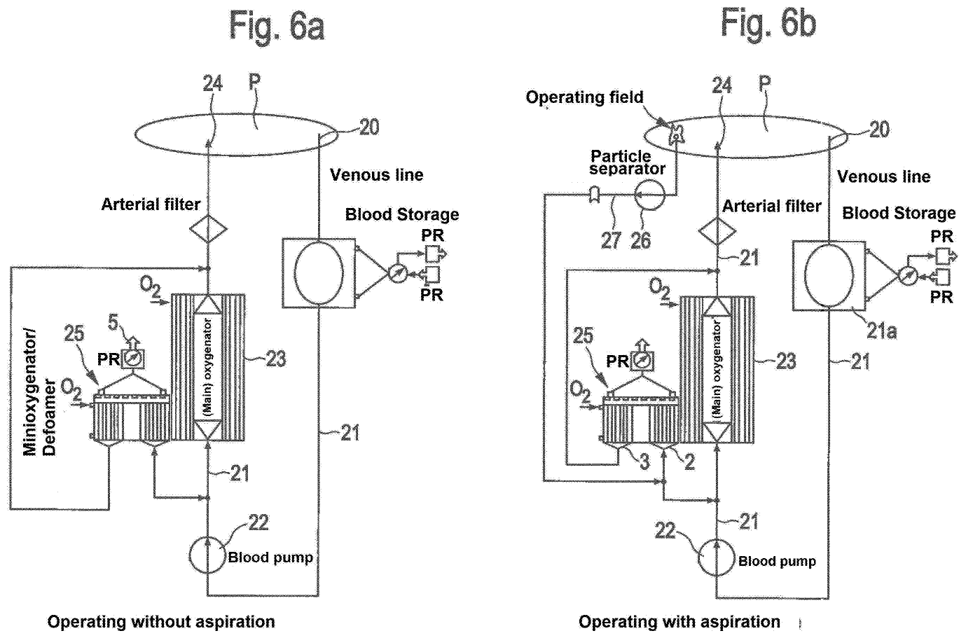

FIGS. 6a and 6b show a possible system that can be used when carrying out surgery on a creature, such as a human. [The system comprises]

The system comprises a blood-drawing implement, for example a cannula 20 through which venous blood is withdrawn from the patient P and conducted in a blood-conducting tube 21 through a blood pump 22 and an oxygenator 23 to a blood-return implement, for example another cannula 24. In this way, a blood circuit is formed for oxygenating the pumped blood.

A subflow of the blood is withdrawn from the tube 21 upstream of the oxygenator 23 and conducted through the degassing apparatus according to the invention that is denoted here generally by reference numeral 25 and corresponds to one of the apparatuses described with respect to FIGS. 1 to 5, even though the apparatus according to FIG. 5 is shown here.

The degassed blood is returned into the tube 21 here, and more particularly, in this embodiment, downstream of the oxygenator. The degassing apparatus thus forms a bypass and blood flows through it continuously.

FIG. 6a shows an application in which the degassing apparatus 25 is not evacuated by the vacuum pump 5. It is not connected to the subatmospheric-pressure chamber, for example.

FIG. 6b shows an application in which blood is drawn off from a surgical site using a blood pump 26 and an inflow line 27 that can thus contain gas bubbles of ambient air. This blood is conducted via the inflow line 27 into the blood inlet 2 of the degassing apparatus 25 and is there stripped of gas bubbles, whereupon the blood is conducted out of the blood outlet 3 downstream of the oxygenator into the tube 21. The degassed blood is thus conducted past the oxygenator.

Only one blood pump 22 is required in the circuit of the tube 21 for this embodiment, since the bypass flow through the degassing apparatus 25 is achieved by the pressure drop across the oxygenator 23.

Also, a blood-storing reservoir 21a can be configured in the tube 21 in the system; however this is not essential for the invention.

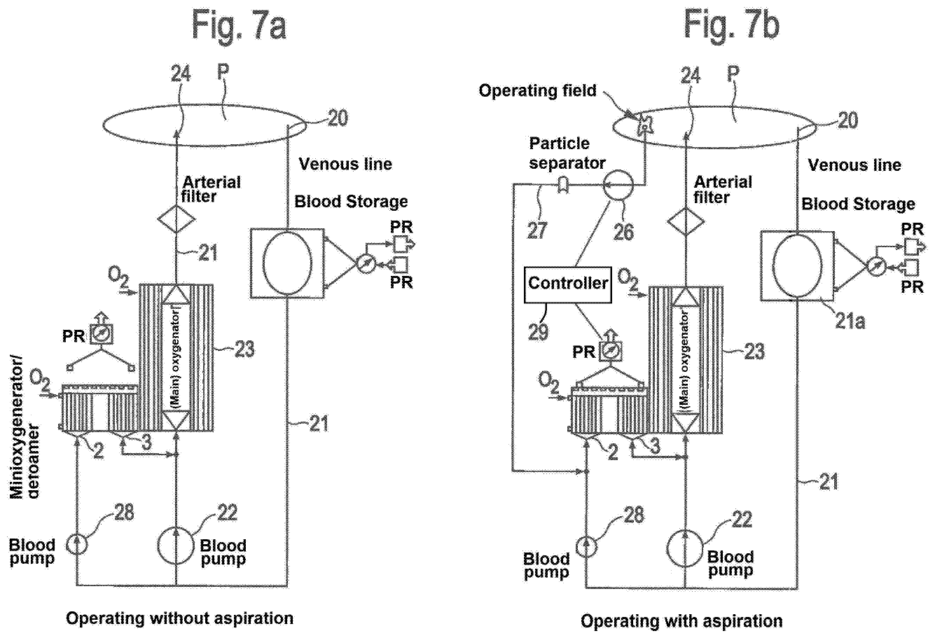

FIGS. 7a and 7b show a system like that of FIG. 6, however with the difference that the subflow of the blood conducted in the circuit that is conducted through the degassing apparatus 25 is returned back into the tube 21 downstream of the degassing apparatus 25, upstream of the oxygenator 23. For this reason, the subflow through the degassing apparatus has to be pumped through the degassing apparatus 25 using an additional blood pump 28, but offers the advantage that the degassed blood can also be enriched with oxygen by the oxygenator 23.

FIG. 7a again shows the application in which the degassing apparatus is only operated in the partial through-flow, without subatmospheric pressure in the subatmospheric-pressure chamber being switched on. FIG. 7b shows the blood being pumped off the surgical site, as in FIG. 6b. Accordingly, the aspirated blood containing bubbles is conducted via the inflow line 27 into the blood inlet 2 of the degassing apparatus 25 here as well.

FIG. 7b also shows an embodiment in which a controller 29 is provided that can operate the blood pump 26 for aspirating blood from the surgical site and the vacuum pump 5 for generating the subatmospheric pressure in the subatmospheric-pressure chamber of the degassing apparatus 25 independently of one another.

Such a controller can be configured in such a way that the blood pump 26 for example does not start to suction, that is, does not start to operate, until the vacuum pump 5 has already started to operate and a subatmospheric pressure for eliminating bubbles has already been reliably formed in the degassing apparatus. The controller can also cause the vacuum pump to only stop operating when the blood pump 26 is switched off and is no longer pumping. Other operating dependencies can also be stored in this regard. Such a controller can also be provided in the system according to FIG. 6; however this is not shown there.

1. A degassing apparatus for removing dissolved gas or gas bubbles from blood, the apparatus comprising:
a blood chamber having a blood inlet and a blood outlet conducting blood conducted through the blood chamber;
a subatmospheric-pressure chamber, having a subatmospheric-pressure connection;
means connected to the connection for evacuating the subatmospheric-pressure chamber through the connection; and
a semipermeable membrane between the subatmospheric-pressure chamber and the blood chamber that is in particular permeable to gas and impermeable to blood, the blood chamber having an upstream and a downstream subchamber that are next to one another in a direction perpendicular to the direction of gravity; and
a bridging subchamber that is part of the subatmospheric-pressure chamber and that connects the upstream and downstream subchambers at upper ends thereof, the blood inlet being in a lower region of the upstream subchamber, and the blood outlet being in a lower region of the downstream subchamber.

2. The degassing apparatus according to claim 1, wherein the subchambers are relatively oriented such that the blood in the upstream subchamber flows at least on average parallel counter to the direction of gravity and, in the downstream subchamber, flows at least on average parallel in the direction of gravity.

3. The degassing apparatus according to claim 1, wherein a subregion of an upper wall region of the bridging subchamber is formed by a semipermeable flat membrane whose one side faces into the blood chamber and whose opposite side faces away from the subatmospheric-pressure chamber.

4. The degassing apparatus according to claim 1, further comprising:
a gassing device generating oxygen bubbles at the blood inlet of the blood chamber.

5. The degassing apparatus according to claim 1, further comprising:
a bundle of semipermeable fiber tubes in the blood chamber and having outer surfaces contacted by blood, and interiors connected to or forming a part of one of the subatmospheric-pressure chambers.

6. The degassing apparatus according to claim 1, further comprising:
a bundle of semipermeable fiber tubes in the subatmospheric-pressure chamber and having outer surfaces acted upon by subatmospheric pressure and interiors contacted by blood, the interiors of the fiber tubes forming at least a portion of the blood chamber.

7. The degassing apparatus according to claim 1, further comprising:
an oxygen supply opening into the subatmospheric-pressure chamber that in particular is configured to supply the subatmospheric-pressure chamber continuously with oxygen, while maintaining a subatmospheric pressure.

8. A system for the extracorporeal treatment of blood, comprising:
a blood-drawing implement;
a blood-return implement;
a blood-conducting tube;
a blood pump;
an oxygenator between the implements such that blood can be pumped through the oxygenator by the blood pump; and
a degassing apparatus according to claim 1 whose blood outlet opens into the blood-conducting tube between the implements, the system being configured to conduct a subflow of the overall blood flowing between the implements through the degassing apparatus.

9. The system according to claim 8, further comprising:
an inflow line having a second blood pump that aspirates blood loaded with gas bubbles from a surrounding area of a surgical site, the inflow line opening into the blood inlet of the degassing apparatus.

10. The system according to claim 8, wherein the blood outlet of the degassing apparatus opens into a blood inlet of the oxygenator.

11. The system according to claim 8, further comprising:
means for generating subatmospheric pressure in the subatmospheric pressure chamber of the degassing apparatus as a function of the operation of the second blood pump in the inflow line such that a subatmospheric pressure is automatically generated in the subatmospheric-pressure chamber as soon as, or before, the second blood pump starts to operate and an ambient pressure s formed in the subatmospheric-pressure chamber as soon as, or after the second blood pump has stopped operating.
